(19) United States
(12) Patent Application Publication
(10) Pub. No.: US 2012/0202297 A1
(43) Pub. Date: Aug. 9, 2012

(54) GENDER DETERMINATION METHOD

(75) Inventors: Constance M. Hendrickson, Irving, TX (US); Denise L. Merkle, Fort Worth, TX (US); John Spurgeon, Whitesboro, TX (US); Gina Spurgeon, Whitesboro, TX (US)

(73) Assignee: HELLO BABY F.S.T. LLC, Whitesboro, TX (US)

(21) Appl. No.: 13/367,944

(22) Filed: Feb. 7, 2012

Related U.S. Application Data
(60) Provisional application No. 61/440,622, filed on Feb. 8, 2011.

(51) Int. Cl. G01N 33/566; G01N 21/78

(52) U.S. Cl. 436/501

(57) ABSTRACT

A non-invasive gender determination method and kits for accomplishing the same are disclosed herein. The present invention is based on difference in testosterone levels in the maternal body fluids of females carrying male fetuses relative to that of females carrying a female fetus. The present invention does not need specialized equipment and can quickly and rapidly detect the presence of testosterone in maternal urine or serum and thus indicate fetal gender. The kits and methods disclosed herein are useful for breeders of single-fetus mammals, veterinarians, and interested parents.

### CROSS-REFERENCE TO RELATED APPLICATIONS

This application claims priority to U.S. Provisional Application Ser. No. 61/440,622, filed Feb. 8, 2011, the entire contents of which are incorporated herein by reference.

### TECHNICAL FIELD OF THE INVENTION

The present invention relates in general to the field of gender determination, and more particularly, to a novel gender determination method using maternal serum or urine.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with gender determination methods.

U.S. Pat. No. 4,840,914 issued to Weisberg (1989) provides a gender-indicating test of the unborn child. The Weisberg invention describes a colorimetric test on the pregnancy urine performed on samples obtained after about the 20th week of the pregnancy. The gender-indicating composition for use in the test is a mixture of alkali hydroxide and metallic aluminum. The colored results of the exothermic reaction of the composition with the urine is evaluated—tan solutions indicate a female child and brown solutions indicate a male child. The Weisberg invention includes the method, the compositions used in the method and a convenient kit containing the subdivided composition in test units for performing the gender-indicating test.

U.S. Patent Application No. 20080108071 (Thompson, 2008) provides non-invasive methods for determining the sex of a human fetus and predicting other genetic abnormalities. The methods include screening a maternal sample for biomarkers known to be associated with risk of genetic abnormalities; removing all or substantially all nucleated and anucleated cell populations from the maternal sample to obtain a remaining material; detecting in the remaining material, the presence of nucleic acid; and determining the sex of the fetus from the nucleic acid wherein the presence of a certain marker is indicative of a male fetus; performing an ultrasound scan which yields quantitative measurements of the fetus; and interpreting the results of the genetic abnormality screening in conjunction with the ultrasound measurements.

U.S. Patent Application No. 20090317817 (Oeth et al. 2009) discloses compositions, processes and kits for noninvasive, early determination of fetal sex from, and/or amount of fetal nucleic acid in, an extracellular nucleic acid sample (blood, plasma, serum) from a pregnant female. Such compositions, processes and kits are useful for detection of low genomic copy numbers of male fetal nucleic acid in a high copy number background of female nucleic acid, thereby determining the sex of a fetus and/or amount of fetal nucleic acid in a sample.

### SUMMARY OF THE INVENTION

The present invention describes a novel, easy-to-use and rapid gender determination method for single-fetus mammals. The method of the present invention is based in the detection of the difference in testosterone levels in the maternal body fluids of females carrying male fetuses relative to those of females carrying a female fetus levels.

In one embodiment the instant invention provides a testing kit for non-invasive determination of fetal gender comprising: (i) a first solid substrate, a test strip, a test stick, a slide or combinations thereof comprising one or more antibodies or beads comprising the one or more antibodies, wherein the antibody is capable of reacting with an antigen when contacted with a maternal biological sample thereby determining the fetal gender, wherein the one or more antibodies or the beads are coupled to the first solid substrate, the test strip, the slide or combinations thereof; (ii) a second solid substrate, a test strip, a test stick, a slide or combinations thereof comprising uncoupled beads, wherein the beads are coupled to the first solid substrate, the test strip, the slide or combinations thereof; and (iii) a manual, a booklet, a leaflet or any combinations thereof detailing usage instructions of the testing kit.

The maternal biological sample that is used in the testing kit of the present invention is selected from the group consisting of serum, urine, blood, and plasma. In one aspect of the present invention the antigen in the maternal biological sample is a male gender specific hormone, wherein the hormone is testosterone. In another aspect the antibody conjugated to the beads is an anti-testosterone antibody (antiT).

In yet another aspect presence of testosterone in the maternal biological results in a shaggy or a furry appearance on the surface of the first solid substrate, the test strip, the test stick, the slide or combinations thereof, wherein the shaggy or furry appearance may be observed using the one or more visualization tools or devices, wherein the one or more visualization tools or devices comprise a microscope, a magnifying glass, a loupe or combinations thereof, wherein the visualization tools or devices may be a part of the kit or may be provided separately. In another aspect the presence of a shaggy or furry appearance is indicative of a male fetus and the absence of a shaggy or furry appearance is indicative of a female fetus.

In another embodiment the present invention provides a method for non-invasive determination of fetal gender comprising the steps of: (i) providing a maternal biological sample, wherein the maternal biological sample is selected from the group consisting of serum, urine, blood, and plasma; (ii) providing a testing kit comprising: a) a first solid substrate, a test strip, a test stick, a slide or combinations thereof comprising one or more antibodies or beads comprising the one or more antibodies, wherein the one or more antibodies or the beads are coupled to the first solid substrate, the test strip, the slide or combinations thereof, b) a second solid substrate, a test strip, a test stick, a slide or combinations thereof comprising uncoupled beads, wherein the beads are coupled to the first solid substrate, the test strip, the slide or combinations thereof, and c) a manual, a booklet, a leaflet or any combinations thereof detailing usage instructions of the testing kit; (iii) contacting the biological sample with the a first solid substrate, a test strip, a test stick, a slide or combinations thereof, wherein the antibody is capable of reacting with an antigen in the maternal biological sample thereby determining the fetal gender; and (iv) observing for a change in appearance on the surface of the first solid substrate, the test strip, the test stick, the slide or combinations thereof, wherein a shaggy or a furry appearance on the surface is indicative of a male fetus and an absence of a shaggy or furry appearance is indicative of a female fetus.

In one aspect of the method disclosed hereinabove the antigen in the maternal biological sample is a male gender specific hormone, wherein the hormone is testosterone. In another aspect the antibody conjugated to the beads is an anti-testosterone antibody (antiT). In yet another aspect the furry or shaggy appearance may be visualized using one or more one or more visualization tools or devices comprising a microscope, a magnifying glass, a loupe or combinations thereof, wherein the visualization tools or devices may be a part of the kit or may be provided separately.

Yet another embodiment of the present invention relates to a testing kit for non-invasive determination of fetal gender comprising: a first solid substrate, a test strip, a test stick, a slide or combinations thereof comprising a first testing reagent comprising one or more antibodies or beads comprising the one or more antibodies and a second testing reagent comprising the one or more antibodies conjugated with a colored dye, wherein the one or more antibodies is capable of reacting with an antigen when contacted with a maternal biological sample leading to a change in a color of the colored dye thereby determining the fetal gender, wherein the one or more antibodies or the beads are coupled to the first solid substrate, the test strip, the slide or combinations thereof, a second solid substrate, a test strip, a test stick, a slide or combinations thereof comprising uncoupled beads, antibodies coupled with a colored dye or combinations thereof, wherein the beads are coupled to the first solid substrate, the test strip, the slide or combinations thereof, and a manual, a booklet, a leaflet or any combinations thereof detailing usage instructions of the testing kit.

In one aspect the maternal biological sample is selected from the group consisting of serum, urine, blood, and plasma. In another aspect the antigen in the maternal biological sample is a male gender specific hormone, wherein the hormone is testosterone. In yet another aspect the antibody conjugated to the beads is an anti-testosterone antibody (antiT). In another aspect a presence of testosterone in the maternal biological results in bleaching of the color of the dye from red to white on the surface of the first solid substrate, the test strip, the test stick, the slide or combinations thereof. The bleaching of the color may be observed one or more visualization tools or devices that may comprise a microscope, a magnifying glass, a loupe or combinations thereof The visualization tools or devices may be a part of the kit or may be provided separately. In one aspect the bleaching of the color from red to white is indicative of a male fetus and the retention of the red color or change to any color other that white is indicative of a female fetus. In a specific aspect the colored dye is phycoerythrin.

In one embodiment the instant invention discloses a method for non-invasive determination of fetal gender comprising the steps of: (i) providing a maternal biological sample, wherein the maternal biological sample is selected from the group consisting of serum, urine, blood, and plasma; (ii) providing a testing kit comprising: a) first solid substrate, a test strip, a test stick, a slide or combinations thereof comprising a first testing reagent comprising one or more antibodies or beads comprising the one or more antibodies and a second testing reagent comprising the one or more antibodies conjugated with a colored dye, wherein the one or more antibodies or the beads are coupled to the first solid substrate, the test strip, the slide or combinations thereof, b) a second solid substrate, a test strip, a test stick, a slide or combinations thereof comprising uncoupled beads, antibodies coupled with a colored dye or combinations thereof, wherein the beads are coupled to the first solid substrate, the test strip, the slide or combinations thereof, and c) a manual, a booklet, a leaflet or any combinations thereof detailing usage instructions of the testing kit; (iii) contacting the biological sample with the a first solid substrate, a test strip, a test stick, a slide or combinations thereof, wherein the one or more antibodies are capable of reacting with an antigen in the maternal biological sample leading to a change in a color thereby determining the fetal gender; and (iv) observing for a change in color from red to colorless on the surface of the first solid substrate, the test strip, the test stick, the slide or combinations thereof, wherein the change from red to white on the surface is indicative of a male fetus and a retention of the red color or a change to a color other than white is indicative of a female fetus.

In a specific aspect of the testing method disclosed hereinabove the antigen in the maternal biological sample is a male gender specific hormone, wherein the hormone is testosterone. In one aspect the antibody conjugated to the beads is an anti-testosterone antibody (antiT). In another aspect the colored dye is phycoerythrin. In yet another aspect the change in the color may be visualized using one or more one or more visualization tools or devices comprising a microscope, a magnifying glass, a loupe or combinations thereof, wherein the visualization tools or devices may be a part of the kit or may be provided separately.

Another embodiment of the instant invention describes a testing kit for non-invasive determination of fetal gender in a mammal comprising: a) a tube, vial or any other suitable container for receiving one or more maternal biological samples; b) a tube or a vial comprising a testing reagent, wherein the testing reagent comprises one or more beads conjugated with an antibody, wherein the antibody is capable of reacting with an antigen in the maternal biological sample thereby determining the fetal gender; c) one or more vials comprising testing controls, wherein the testing controls comprise one or more conjugated beads in a buffer, saline or water, one or more unconjugated beads with the antigen or both; d) one or more slides or other solid substrates for reacting the maternal biological sample with the testing reagents and the testing controls; and e) a manual, a booklet, a leaflet or any combinations thereof detailing usage instructions of the testing kit.

In one aspect the testing kit is used along with one or more visualization tools or devices (capable of providing at least a 100× magnification) comprising a microscope, a magnifying glass, a loupe or combinations thereof, wherein the visualization tools or devices may be a part of the kit or may be provided separately. In another aspect the maternal biological sample is selected from the group consisting of serum, urine, blood, and plasma. In a specific aspect the antigen in the maternal biological sample is a male gender specific hormone, wherein the hormone is testosterone. In yet another aspect the antibody conjugated to the beads is an anti-testosterone antibody (antiT).

In the testing kit disclosed hereinabove a presence of testosterone in the maternal biological sample leads to a clumping of the one or more conjugated beads on the slide or the solid substrate, wherein the clumping is observed using the one or more visualization tools or devices. Clumping of the one or more conjugated beads is indicative of a male fetus, and conversely an absence clumping of the one or more conjugated beads is indicative of a female fetus. In another aspect the mammal is a single-fetus human or animal.

Yet another embodiment of the present invention relates to a method for determining fetal gender in a mammal comprising the steps of: i) providing a testing kit comprising: a tube, vial or any other suitable container for receiving one or more maternal biological samples, wherein the biological sample comprises blood, serum, urine, plasma or combinations thereof, a tube or a vial comprising a testing reagent, wherein the testing reagent comprises one or more beads conjugated with an antibody, wherein the antibody is capable of reacting with an antigen in the maternal biological sample thereby determining the fetal gender, one or more vials comprising testing controls, wherein the testing controls comprise one or more conjugated beads in a buffer, saline or water, one or more unconjugated beads with the antigen or both, one or more slides or other solid substrates for reacting the maternal biological sample with the testing reagents and the testing controls, and a manual, a booklet, a leaflet or any combinations thereof detailing usage instructions of the testing kit; ii) obtaining the one or more maternal biological samples; iii) adding the testing reagent to a first region or portion of the slide or the solid substrate; iv) adding the one or more biological samples to the first region or portion of the slide or substrate comprising the testing reagent; v) adding the one or more testing controls to a second region or portion of the slide or the solid substrate; vi) adding the one or more biological samples to the second region or portion of the slide or substrate comprising the testing controls; and vii) observing for a presence or absence of clumping in the first and second regions or portions of the slide or substrate.

In one aspect an observation for the presence or absence of clumping is performed using one or more visualization tools or devices comprising a microscope, a magnifying glass, a loupe or combinations thereof, wherein the visualization tools or devices may be a part of the kit or may be provided separately and provide at least a 100x magnification. In a specific aspect the antigen in the maternal biological sample is a male gender specific hormone, wherein the hormone is testosterone. In another aspect the antibody conjugated to the beads is an anti-testosterone antibody (antiT). In yet another aspect the presence of clumping in the first region or portion and an absence of clumping in the second region or portion of the slide or substrate is indicative of a male fetus. In another aspect the absence of clumping in the first and second regions or portions of the slide or substrate is indicative of a female fetus. In another aspect the presence of clumping in the second region or portion of the slide or substrate is indicative of a false positive, a testing error or a sample or kit contamination. In a related aspect the mammal is a single-fetus human or animal. In one aspect a reaction of the testing reagent and the sample and the reaction of the testing control and the sample may be performed on separate slides or substrates. In another aspect the method may be used to confirm gender determination results done by invasive or non-invasive techniques selected from the group consisting of ultrasound, diagnostic kits, real-time qPCR of maternal plasma, sonography, amniocentesis, chorionic villus sampling (CVS), and any combinations thereof

The present invention further discloses a testing kit for non-invasive determination of fetal gender in a mammal comprising: (a) a tube, vial or any other suitable container for receiving one or more maternal biological samples; (b) a tube or a vial comprising a first testing reagent, wherein the first testing reagent comprises one or more beads conjugated with an antibody, wherein the antibody is capable of binding with an antigen in the maternal biological sample; (c) a tube or a vial comprising a second testing reagent, wherein the second testing reagent comprises the antibody conjugated with a colored dye, wherein the colored dye is capable of being bleached following reaction of the antibody with the antigen in the maternal biological sample thereby determining the fetal gender; (d) one or more vials comprising testing controls, wherein the testing controls comprise one or more conjugated beads in a buffer, saline or water, one or more unconjugated beads with the antigen, and or more conjugated beads in a buffer, saline or water with the antibody conjugated with the dye; (e) one or more slides or other solid substrates for reacting the maternal biological sample with the testing reagents and the testing controls; and (f) a manual, a booklet, a leaflet or any combinations thereof detailing usage instructions of the testing kit.

In one aspect of the kit disclosed hereinabove the kit is used along with one or more visualization tools or devices comprising a microscope, a magnifying glass, a loupe or combinations thereof, wherein the visualization tools or devices may be a part of the kit or may be provided separately. In another aspect the visualization tools or devices provide at least a 100x magnification. In yet another aspect the maternal biological sample is selected from the group consisting of serum, urine, blood, and plasma. In a specific aspect the antigen in the maternal biological sample is a male gender specific hormone, wherein the hormone is testosterone. In another aspect the antibody conjugated to the beads is an anti-testosterone antibody (antiT). In related aspects presence of testosterone in the maternal biological sample leads to a bleaching of a color of the dye coated antibody from red to white on the slide or the solid substrate, wherein the bleaching is observed using the one or more visualization tools or devices and a retention of the red color or a change to a color other than white is indicative of a female fetus. In another aspect the mammal is a single-fetus human or animal.

In one embodiment the present invention provides a method for determining fetal gender in a mammal comprising the steps of: i) providing a testing kit comprising: a) a tube, vial or any other suitable container for receiving one or more maternal biological samples, wherein the biological sample comprises blood, serum, urine, plasma or combinations thereof, b) a tube or a vial comprising a first testing reagent, wherein the first testing reagent comprises one or more beads conjugated with an antibody, wherein the antibody is capable of binding with an antigen in the maternal biological sample, c) a tube or a vial comprising a second testing reagent, wherein the second testing reagent comprises the antibody conjugated with a colored dye, wherein the colored dye is capable of being bleached following reaction of the antibody with the antigen in the maternal biological sample thereby determining the fetal gender, d) one or more vials comprising testing controls, wherein the testing controls comprise one or more conjugated beads in a buffer, saline or water, one or more unconjugated beads with the antigen, and or more conjugated beads in a buffer, saline or water with the antibody conjugated with the dye, e) one or more slides or other solid substrates for reacting the maternal biological sample with the testing reagents and the testing controls, and f) a manual, a booklet, a leaflet or any combinations thereof detailing usage instructions of the testing kit; ii) obtaining the one or more maternal biological samples; iii) adding the first testing reagent to a first region or portion of the slide or the solid substrate; iv) adding the one or more biological samples to the first region or portion of the slide or substrate comprising the first testing reagent to form a mixture; v) incubating the mixture for a specified period of time; vi) adding the second testing reagent to the first region or portion of the slide comprising the mixture; vii) adding the one or more testing controls to a second region or portion of the slide or the solid substrate; viii) adding the one or more biological samples to the second region or portion of the slide or substrate comprising the testing controls to form a mixture; ix) adding the second testing reagent to the second region or portion of the slide comprising the mixture; and x) observing for a bleaching of color of the second testing reagent in the first and second regions or portions of the slide or substrate.

In one aspect of the method described hereinabove an observation for of the bleaching of color of the second testing reagent is performed using one or more visualization tools or devices comprising a microscope, a magnifying glass, a loupe or combinations thereof, wherein the visualization tools or devices may be a part of the kit or may be provided separately. In another aspect the visualization tools or devices provide at least a 100× magnification. In a specific aspect the antigen in the maternal biological sample is a male gender specific hormone, wherein the hormone is testosterone. In yet another aspect the antibody conjugated to the beads is an anti-testosterone antibody (antiT).

In related aspects the bleaching of color from red to white of the second testing reagent in the first region or portion and a retention of a red color of the second testing reagent in the second region or portion of the slide or substrate is indicative of a male fetus, the retention of the red color in the first and second regions or portions of the slide or substrate is indicative of a female fetus and the bleaching of color from red to white of the second testing reagent in the second region or portion of the slide or substrate is indicative of a false positive, a testing error or a sample or kit contamination. In another aspect the mammal is a single-fetus human or animal.

In yet another aspect a reaction of the testing reagent and the sample and the reaction of the testing control and the sample may be performed on separate slides or substrates. In another specific aspect the colored dye is phycoerythrin. In another aspect the method may be used to confirm gender determination results done by invasive or non-invasive techniques selected from the group consisting of ultrasound, diagnostic kits, real-time qPCR of maternal plasma, sonography, amniocentesis, chorionic villus sampling (CVS), and any combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 is a microscopic image of maternal urine at 100x magnification showing no clumping;

FIG. 2 shows a microscopic image of testosterone spiked maternal urine at 200× magnification showing point clumping;

FIG. 3 is a microscopic image of maternal urine (pregnant with a male fetus) at 200× magnification;

FIG. 4 is a microscopic image of maternal urine (pregnant with a male fetus) at 200× magnification showing clumping and coverage with the protein;

FIG. 5 is a microscopic image of maternal urine (pregnant with a male fetus) at 400× magnification showing clumping and coverage with the protein; and

FIG. 6 shows one embodiment of a test strip 10 in which the sample is loaded at opening 12.

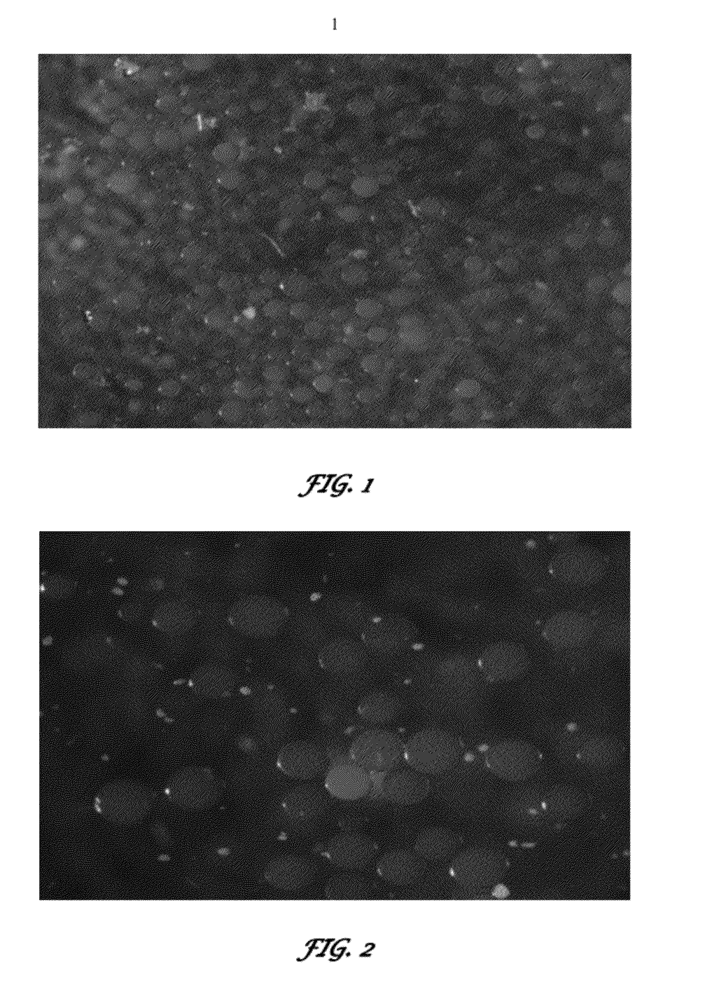

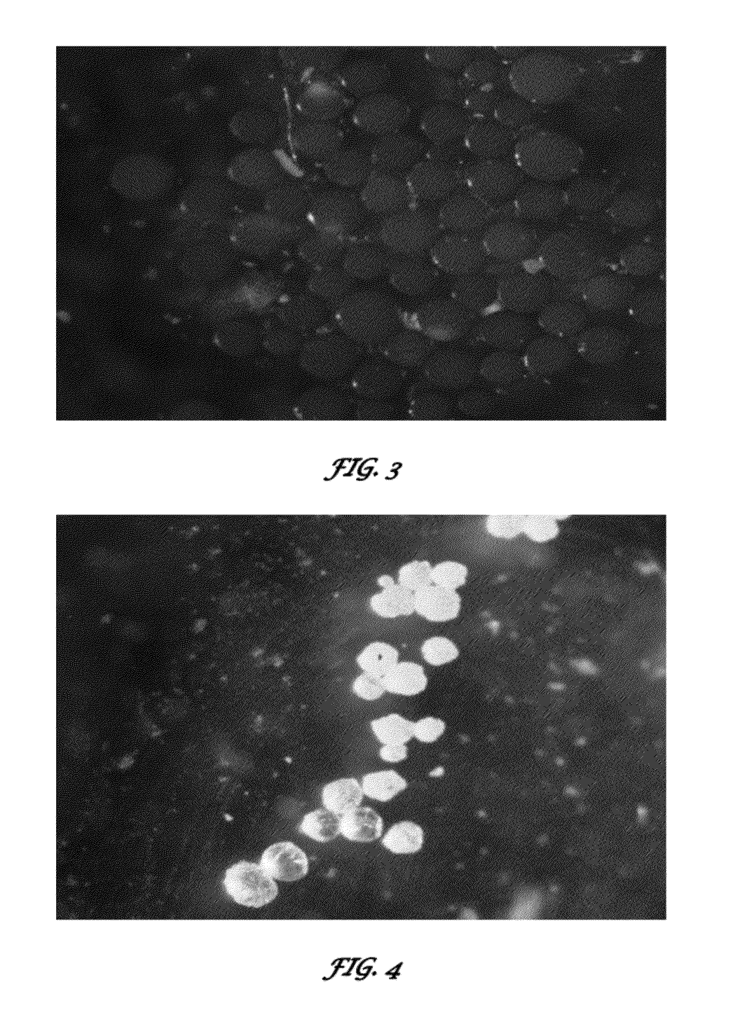

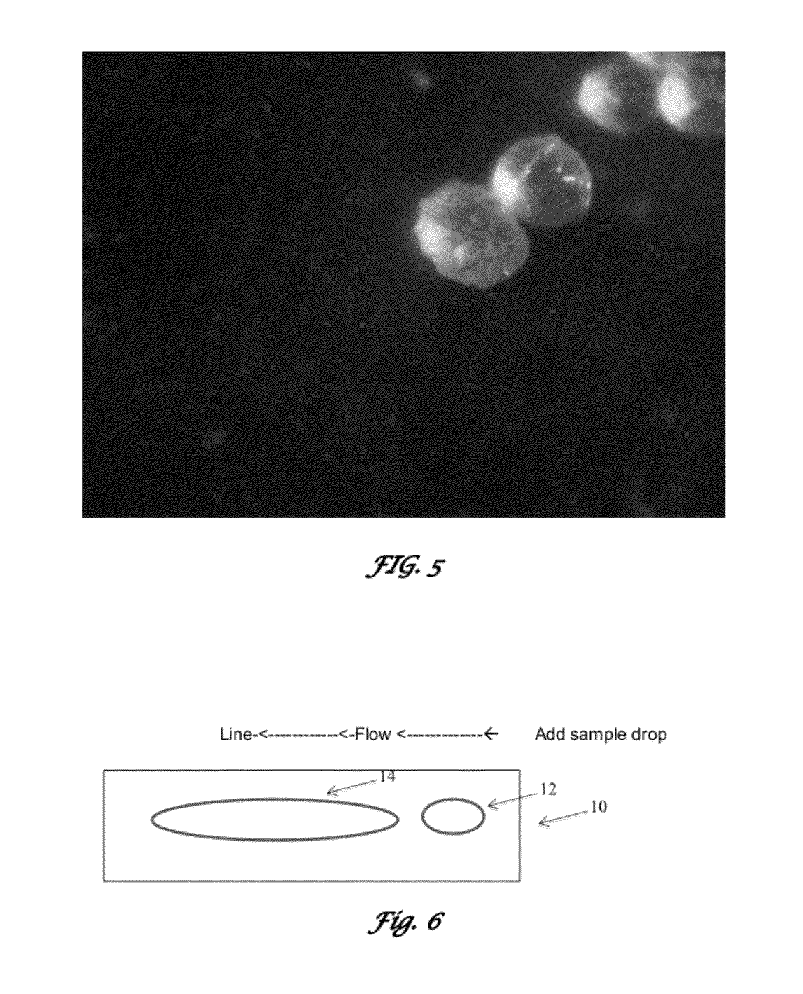

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below.

Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

The term “test kit” or “testing kit” denotes combinations of reagents and adjuvants required for an analysis. Although a test kit consists in most cases of several units, one-piece analysis elements are also available, which must likewise be regarded as test kits.

The term “fetus” as used herein in the specification and claims refers to any prenatal organism between conception and birth which is normally developed in utero. This definition also includes a prenatal organism which is first conceived in vitro and later implanted in a uterus. The term “fetus” also includes the term “embryo.”

The term “serum” refers to the cell-free liquid portion of blood remaining after clot formation and removal (factors involved in clotting are removed, i.e., fibrin). The term “plasma” refers to the cell-free liquid portion of blood which has been prevented from clotting by the use of anti-coagulants (clotting factors still remain). As used herein, the term “urine” refers to all forms of nitrogen-rich waste processed by the kidneys of an animal.

As used herein, the term “antigen” is used in reference to any substance that is capable of being recognized by an antibody. It is intended that this term encompass any “antigen” and “immunogen” (i.e., a substance which induces the formation of antibodies).

The term “antibody” (or “antibodies”) is used herein in the broadest sense and refers to intact molecules as well as fragments thereof, which binds specifically to an antigen or an antigenic determinant, and specifically, binds to proteins identical or structurally related to the antigenic determinant which stimulated their production. Thus, antibodies are useful in assays to detect the antigen, which stimulated their production.

As used herein, the term “hormone” refers to a chemical substance, released from a living cell into the extracellular fluid in low quantities, which acts on a target cell to produce a response. Hormones are classified on the basis of chemical structure; most hormones are polypeptides, steroids or derived from a single amino acid (Kirk-Othmer “Concise Encyclopedia of Chemical Technology”, John Wiley & Sons, Inc., 4th Edition (1999), p. 1055). Steroid hormones include estrogens, androgens (e.g., testosterone and derivatives thereof), corticoids and progestins.

The terms “testosterone” refers to the naturally occurring hormone known as testosterone having the chemical name 17-β-hydroxyandrost-4-en-3-one which may be isolated and purified from nature or synthetically produced in any manner. The terms also comprises pharmaceutically acceptable esters, i.e., compounds where the “H” of the “OH” group is replaced with an alkyl group, e.g. propionate, cypionate and enanthate and other derivatives including methyltestosterone, methandrostenolone, fluovymesterone, and danazol. A number of pharmaceutically useful derivatives of testosterone, which are intended to be encompassed by the term testosterone as used here are disclosed within the Physician's Desk Reference (most recent edition) as well as Harrison's Principles of Internal Medicine. In addition, applicants refer to U.S. Pat. No. 5,536,714 issued Jul. 16, 1996; U.S. Pat. No. 5,824,668 issued Oct. 20, 1998; U.S. Pat. No. 3,980,638 issued Sep. 14, 1996; U.S. Pat. No. 4,031,117 issued Jun. 21, 1977; U.S. Pat. No. 4,085,202 issued Apr. 18, 1978; U.S. Pat. No. 4,197,286 issued Apr. 8, 1980; U.S. Pat. No. 4,507,290 issued Mar. 26, 1985 and U.S. Pat. No. 5,622,944 issued Apr. 22, 1997 all of which are incorporated herein by reference to disclose and describe testosterone derivatives and formulations.

A novel, non-invasive, easy-to-use, and rapid gender determination method for single fetus mammals is disclosed herein. The method of the present invention is based on detecting the presence of the hormone testosterone in maternal urine or serum samples and thereby indicating fetal gender.

The determination of gender remains a significant medical and veterinary concern, notably in obstetrics and animal breeding. Human fetal gender is almost always of interest to expectant parents, and is the subject of many imaging and hematological tests. In the veterinary sphere, the financial pressures of owning and raising valuable mammals also spur owners and breeders to determine fetal gender.

Gender is dictated chromosomally but is also dependent on hormone ratios. Both males and females require testosterone for normal reproductive development and function, however male characteristics result from a much higher level of testosterone than estrogen in the body (˜98% in adults). Testosterone, therefore, is an historical and useful measure of the presence of a Y chromosome.

Clinical Laboratory and Imaging Tests are commonly used to determine fetal gender. While highly accurate, these analyses involve specialized equipment, trained personnel, and long lead times—or all three. For e.g., sonography of pregnant horses is only possible for a very narrow window of time, and carries the risk of rupture, infection, miscarriage, etc.

It has been demonstrated that the existence of a male fetus correlates to the presence of detectable testosterone in maternal urine or serum, whereas the presence in utero of a female fetus does not.

The significant difference in testosterone levels in the maternal body fluids of females carrying male fetuses relative to that of females carrying a female fetus permitted the development of a novel test for gender typing of a fetus. Without requiring specialized equipment, the new test described herein quickly detects the presence of testosterone in maternal urine or serum, and indicates fetal gender. A simple kit or set of reagents can be combined for the use of veterinarians, breeders, humans with fetuses, etc.

Method 1

Aggregation as an Indication of the Presence of Significant Testosterone

An aliquot of Anti-testosterone antibody (antiT) CNBr-conjugated-Sepharose 6B beads is placed into the well of a microscope slide or well plate. An appropriate amount of sample (serum, urine, etc.) by volume is added. The mixture is immediately observed via optics of at least 100 power, such as a microscope, magnifying glass or loupe; one of the latter two are easily included in the kit. In the presence of testosterone the antiT-conjugated beads obviously aggregate and develop a readily identifiable shaggy or furry appearance.

Included Controls: (i) Conjugated beads+saline or other testosterone-free, sample equivalent liquid and (ii) Unconjugated beads+testosterone.

Materials and Methods: Cyanogen bromide activated sepharose beads and testosterone were obtained from Sigma Aldrich. Antitestosterone antibody (aTab) was obtained from Abnova (rabbit polyclonal against testosterone) 10 mg protein per mg sepharose beads.

Buffers and solutions: A. 0.5 M NaHCO₃ containing 0.5 M NaCl pH 8.3-8.5 (coupling buffer); B. cold 1 mM HCl (swelling solution); C. 0.2 M glycine (blocking solution), 0.1 M acetate buffer pH 4 containing 0.4 M NaCl (blocking washout), and 1.0 M NaCl at with sodium azide (storage solution).

(i) The antitestosterone antibody (aTab) was dissolved in buffer A (0.025-0.25 mg/mL); (ii) The resin was swelled in buffer B for 1 hour (200 mL/gm of dry resin); (iii) The supernatant was removed after swirling and wash repeated 2 times for a total of 3 washes; (iv) The resin was then washed first with distilled water and then with buffer A and immediately transferred to a solution of aTab in buffer A; (v) The aTab and resin were mixed for two hours at room temperature on a rocker mixer; (vi) The reacted resin/aTab were then rinsed with buffer A; (vii) The reacted resin/aTab were then rinsed with buffer C in order to block unreacted groups (2 hours at room temperature); (viii) The reacted resin/aTab were then washed extensively with buffer A alternating with buffer D, for a total of 5 cycles; (ix) Storage was in buffer E with 0.02% sodium azide.

Results from Method 1 for three different samples were reacted with resin/aTab are presented in Table 1.

TABLE 1
Results from Method 1 for the different samples.

| Sample | Result |
|---|---|
| No testosterone (distilled water or female urine) | no clumping fine |
| | particles |
| Testosterone purchased from Sigma Aldrich or | visible clumping |
| Neogen (~1.5 μg/L) | |
| Urine sample from pregnant female with | visible clumping |
| confirmed male fetus (> than 4 μg/L) | |

Method 2

Color Bleaching as an Indication of the Presence of Significant Testosterone

An aliquot of Anti-testosterone antibody (antiT) CNBr-conjugated-Sepharose 6B beads is placed into the well of a microscope slide. An appropriate amount of sample (serum or urine) by volume is added. After an incubation time of 1 hour, solution of phycoerythrin-labeled anti-Testosterone (PEantiT) antibody is added. The mixture is immediately observed via optics of at least 100 power, such as a microscope, magnifying glass or loupe; one of the latter two are easily included in the kit. In the presence of testosterone the PEantiT bleaches from red to colorless.

Included Controls: (i) Conjugated beads+saline or other testosterone-free, sample equivalent liquid; (ii) Unconjugated beads+testosterone, and (iii) Conjugated beads+saline+PEantiT.

As mentioned hereinabove, color changes as an indication of the presence of significant testosterone can be used to simplify the visualization of the antibody reaction. The used phytoerythrin-labeled antibody. An aliquot of Anti-testosterone antibody (antiT) CNBr-conjugated-Sepharose beads is placed into the well of a microscope slide. An appropriate amount of sample (serum or urine) by volume is added. After an incubation time of 1 hour, solution of phycoerythrin-labeled anti-Testosterone (PEantiT) antibody is added. The mixture is immediately observed via optics of at least 100 power, such as a microscope, magnifying glass or loupe; one of the latter two are easily included in the kit. In the presence of testosterone the PE anti-T bleaches from red to colorless.

The methods described hereinabove may be useful for breeders of single-fetus mammals, veterinarians, and interested parents.

Method 3

A microsphere agglutination method was developed similar to the original method described in the application, the difference being that the latex beads are colored (blue, red, or black) and visible to the naked eye, and addition of testosterone results in visible pellet formation.

Agglutination/pellet formation: using nanoparticles of colloidal gold coated with testosterone antibody, we have observed, upon the addition of samples containing testosterone, the formation of a dark pellet from aggregated particles:

TABLE 1

| | Sample # | | | | | | |
|---|---|---|---|---|---|---|---|
| | 1 | 2 | 3 | 4 | 5 | 6 | |
| Blank (PBS*) | − | − | − | − | − | − | − |
| PBS spiked with testosterone | + | + | + | + | + | + | |
| 5-30 μg/mL | | | | | | | |
| Urine spiked with testosterone | ++ | + | + | ++ | ++ | + | |
| 5-30 μg/mL | | | | | | | |
| Pregnant urine (human) female fetus | − | − | − | − | − | − | |
| Pregnant urine (human) male fetus | + | ++ | ++ | + | +++ | + | |
| Pregnant serum (horse) filly | − | − | | | | | |
| Pregnant serum (horse) foal | ++ | + | | | | | |

− No precipitate (ppt)
+ small amount of ppt
++ more ppt
+++ large ppt
*phosphate buffered saline

A nylon ribbon treated with the colloidal gold particles coated was also used with testosterone antibody. The coated particles are pipetted onto the ribbon and air-dried. This gives the white ribbon a reddish color. When a drop containing testosterone is pipetted onto the treated ribbon, a large white spot with reddish-black edges appears where the drop is added. If a blank drop is placed on the treated ribbon, there is no change in the coloration.

TABLE 2

| | Sample # | | | | | | |
|---|---|---|---|---|---|---|---|
| | 1 | 2 | 3 | 4 | 5 | 6 | |
| Blank (PBS*) | − | − | − | − | − | − | − |
| PBS spiked with testosterone | + | + | + | + | + | + | |
| 5-30 μg/mL | | | | | | | |
| Urine spiked with testosterone | + | + | + | + | + | + | |
| 5-30 μg/mL | | | | | | | |
| Pregnant urine (human) girl | − | − | − | − | − | − | |
| Pregnant urine (human) boy | + | + | + | + | + | + | |
| Pregnant serum (horse) filly | − | − | | | | | |
| Pregnant serum (horse) foal | + | + | | | | | |

− No change in color
+ White spot
*phosphate buffered saline

The previous method has been used in a lateral flow device, where the sample or urine added “pushes” the aggregated gold particles to the end of the strip, forming a black line. FIG. 6 shows one embodiment of a test strip 10 in which the sample is loaded at opening 12. Upon the interaction between the aggregated gold particles to the end of the strip they form a black line of spot 14. The laminar flow pattern is shown on top of the test strip 10. Not shown, is a cassette (plastic holder such as those used for pregnancy tests) that would encase the test strip 10. Additionally, coated particles may be added in a line across the ribbon, so that a black line is formed when the flowing sample containing testosterone reaches it. This technique may also be utilized in a “dip-stick” format.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof' as used herein refers to all permutations and combinations of the listed items preceding the term. For example, “A, B, C, or combinations thereof' is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 4,840,914: Gender-Indicating Colormetric Test on Pregnancy Urine and Test Kit Therefor.

U.S. Patent Application No. 20080108071: Methods and Systems to Determine Fetal Sex and Detect Fetal Abnormalities.

U.S. Patent Application No. 20090317817: Nucleic Acid-Based Tests for Prenatal Gender Determination.

What is claimed is:

1. A testing kit for non-invasive determination of fetal gender comprising:
a first solid substrate, a test strip, a test stick, a slide or combinations thereof comprising one or more antibodies or beads comprising the one or more antibodies, wherein the antibody is capable of reacting with an antigen when contacted with a maternal biological sample thereby determining the fetal gender, wherein the one or more antibodies or the beads are coupled to the first solid substrate, the test strip, the slide or combinations thereof;
a second solid substrate, a test strip, a test stick, a slide or combinations thereof comprising uncoupled beads, wherein the beads are coupled to the first solid substrate, the test strip, the slide or combinations thereof; and
a manual, a booklet, a leaflet or any combinations thereof detailing usage instructions of the testing kit.

2. The testing kit of claim 1, wherein the maternal biological sample is selected from the group consisting of serum, urine, blood, and plasma.

3. The testing kit of claim 1, wherein the antigen in the maternal biological sample is a male gender specific hormone, wherein the hormone is testosterone.

4. The testing kit of claim 1, wherein the antibody conjugated to the beads is an anti-testosterone antibody (antiT).

5. The testing kit of claim 1, wherein a presence of testosterone in the maternal biological results in a shaggy or a furry appearance on the surface of the first solid substrate, the test strip, the test stick, the slide or combinations thereof, wherein the shaggy or furry appearance may be observed using the one or more visualization tools or devices.

6. The testing kit of claim 5, wherein the one or more visualization tools or devices comprise a microscope, a magnifying glass, a loupe or combinations thereof, wherein the visualization tools or devices may be a part of the kit or may be provided separately.

7. The testing kit of claim 1, wherein a presence of a shaggy or furry appearance is indicative of a male fetus.

8. The testing kit of claim 1, wherein an absence of a shaggy or furry appearance is indicative of a female fetus.

9. A method for non-invasive determination of fetal gender comprising the steps of:
providing a maternal biological sample, wherein the maternal biological sample is selected from the group consisting of serum, urine, blood, and plasma;
providing a testing kit comprising:
a first solid substrate, a test strip, a test stick, a slide or combinations thereof comprising one or more antibodies or beads comprising the one or more antibodies, wherein the one or more antibodies or the beads are coupled to the first solid substrate, the test strip, the slide or combinations thereof;
a second solid substrate, a test strip, a test stick, a slide or combinations thereof comprising uncoupled beads, wherein the beads are coupled to the first solid substrate, the test strip, the slide or combinations thereof; and
a manual, a booklet, a leaflet or any combinations thereof detailing usage instructions of the testing kit;
contacting the biological sample with the a first solid substrate, a test strip, a test stick, a slide or combinations thereof, wherein the antibody is capable of reacting with an antigen in the maternal biological sample thereby determining the fetal gender; and
observing for a change in appearance on the surface of the first solid substrate, the test strip, the test stick, the slide or combinations thereof, wherein a shaggy or a furry appearance on the surface is indicative of a male fetus and an absence of a shaggy or furry appearance is indicative of a female fetus.

10. The method of claim 9, wherein the antigen in the maternal biological sample is a male gender specific hormone, wherein the hormone is testosterone.

11. The method of claim 9, wherein the antibody conjugated to the beads is an anti-testosterone antibody (antiT).

12. The method of claim 9, wherein the furry or shaggy appearance may be visualized using one or more one or more visualization tools or devices comprising a microscope, a magnifying glass, a loupe or combinations thereof, wherein the visualization tools or devices may be a part of the kit or may be provided separately.

13. A testing kit for non-invasive determination of fetal gender comprising:
a first solid substrate, a test strip, a test stick, a slide or combinations thereof comprising a first testing reagent comprising one or more antibodies or beads comprising the one or more antibodies and a second testing reagent comprising the one or more antibodies conjugated with a colored dye, wherein the one or more antibodies is capable of reacting with an antigen when contacted with a maternal biological sample leading to a change in a color of the colored dye thereby determining the fetal gender, wherein the one or more antibodies or the beads are coupled to the first solid substrate, the test strip, the slide or combinations thereof;
a second solid substrate, a test strip, a test stick, a slide or combinations thereof comprising uncoupled beads, antibodies coupled with a colored dye or combinations thereof, wherein the beads are coupled to the first solid substrate, the test strip, the slide or combinations thereof; and
a manual, a booklet, a leaflet or any combinations thereof detailing usage instructions of the testing kit.

14. The testing kit of claim 13, wherein the maternal biological sample is selected from the group consisting of serum, urine, blood, and plasma.

15. The testing kit of claim 13, wherein the antigen in the maternal biological sample is a male gender specific hormone, wherein the hormone is testosterone.

16. The testing kit of claim 13, wherein the antibody conjugated to the beads is an anti-testosterone antibody (antiT).

17. The testing kit of claim 13, wherein a presence of testosterone in the maternal biological results in bleaching of the color of the dye from red to white on the surface of the first solid substrate, the test strip, the test stick, the slide or combinations thereof, wherein the bleaching of the color may be observed one or more visualization tools or devices.

18. The testing kit of claim 17, wherein the one or more visualization tools or devices comprise a microscope, a magnifying glass, a loupe or combinations thereof, wherein the visualization tools or devices may be a part of the kit or may be provided separately.

19. The testing kit of claim 13, wherein the bleaching of the color from red to white is indicative of a male fetus.

20. The testing kit of claim 13, wherein a retention of the red color or change to any color other that white is indicative of a female fetus.

21. The testing kit of claim 13, wherein the colored dye is phycoerythrin.

22. A method for non-invasive determination of fetal gender comprising the steps of:
providing a maternal biological sample, wherein the maternal biological sample is selected from the group consisting of serum, urine, blood, and plasma;
providing a testing kit comprising:
first solid substrate, a test strip, a test stick, a slide or combinations thereof comprising a first testing reagent comprising one or more antibodies or beads comprising the one or more antibodies and a second testing reagent comprising the one or more antibodies conjugated with a colored dye, wherein the one or more antibodies or the beads are coupled to the first solid substrate, the test strip, the slide or combinations thereof;
a second solid substrate, a test strip, a test stick, a slide or combinations thereof comprising uncoupled beads, antibodies coupled with a colored dye or combinations thereof, wherein the beads are coupled to the first solid substrate, the test strip, the slide or combinations thereof; and
a manual, a booklet, a leaflet or any combinations thereof detailing usage instructions of the testing kit.
contacting the biological sample with the a first solid substrate, a test strip, a test stick, a slide or combinations thereof, wherein the one or more antibodies are capable of reacting with an antigen in the maternal biological sample leading to a change in a color thereby determining the fetal gender; and
observing for a change in color from red to colorless on the surface of the first solid substrate, the test strip, the test stick, the slide or combinations thereof, wherein the change from red to white on the surface is indicative of a male fetus and a retention of the red color or a change to a color other than white is indicative of a female fetus.

23. The method of claim 21, wherein the antigen in the maternal biological sample is a male gender specific hormone, wherein the hormone is testosterone.

24. The method of claim 21, wherein the antibody conjugated to the beads is an anti-testosterone antibody (antiT).

25. The method of claim 21, wherein the colored dye is phycoerythrin.

26. The method of claim 21, wherein the change in the color may be visualized using one or more one or more visualization tools or devices comprising a microscope, a magnifying glass, a loupe or combinations thereof, wherein the visualization tools or devices may be a part of the kit or may be provided separately.

27. A testing kit for non-invasive determination of fetal gender in a mammal comprising:
a tube, vial or any other suitable container for receiving one or more maternal biological samples;
a tube or a vial comprising a testing reagent, wherein the testing reagent comprises one or more beads conjugated with an antibody, wherein the antibody is capable of reacting with an antigen in the maternal biological sample thereby determining the fetal gender;
one or more vials comprising testing controls, wherein the testing controls comprise one or more conjugated beads in a buffer, saline or water, one or more unconjugated beads with the antigen or both;
one or more slides or other solid substrates for reacting the maternal biological sample with the testing reagents and the testing controls; and
a manual, a booklet, a leaflet or any combinations thereof detailing usage instructions of the testing kit.

28. The testing kit of claim 27, wherein the testing kit is used along with one or more visualization tools or devices comprising a microscope, a magnifying glass, a loupe or combinations thereof, wherein the visualization tools or devices may be a part of the kit or may be provided separately.

29. The testing kit of claim 28, wherein the visualization tools or devices provide at least a 100× magnification.

30. The testing kit of claim 27, wherein the maternal biological sample is selected from the group consisting of serum, urine, blood, and plasma.

31. The testing kit of claim 27, wherein the antigen in the maternal biological sample is a male gender specific hormone, wherein the hormone is testosterone

32. The testing kit of claim 27, wherein the antibody conjugated to the beads is an anti-testosterone antibody (antiT).

33. The testing kit of claim 27, wherein a presence of testosterone in the maternal biological sample leads to a clumping of the one or more conjugated beads on the slide or the solid substrate, wherein the clumping is observed using the one or more visualization tools or devices.

34. The testing kit of claim 33, wherein the clumping of the one or more conjugated beads is indicative of a male fetus.

35. The testing kit of claim 33, wherein an absence clumping of the one or more conjugated beads is indicative of a female fetus.

36. The testing kit of claim 27, wherein the mammal is a single-fetus human or animal.

37. A method for determining fetal gender in a mammal comprising the steps of:
providing a testing kit comprising:
a tube, vial or any other suitable container for receiving one or more maternal biological samples, wherein the biological sample comprises blood, serum, urine, plasma or combinations thereof;
a tube or a vial comprising a testing reagent, wherein the testing reagent comprises one or more beads conjugated with an antibody, wherein the antibody is capable of reacting with an antigen in the maternal biological sample thereby determining the fetal gender;
one or more vials comprising testing controls, wherein the testing controls comprise one or more conjugated beads in a buffer, saline or water, one or more unconjugated beads with the antigen or both;
one or more slides or other solid substrates for reacting the maternal biological sample with the testing reagents and the testing controls; and
a manual, a booklet, a leaflet or any combinations thereof detailing usage instructions of the testing kit.
obtaining the one or more maternal biological samples;
adding the testing reagent to a first region or portion of the slide or the solid substrate;
adding the one or more biological samples to the first region or portion of the slide or substrate comprising the testing reagent;
adding the one or more testing controls to a second region or portion of the slide or the solid substrate;
adding the one or more biological samples to the second region or portion of the slide or substrate comprising the testing controls; and
observing for a presence or absence of clumping in the first and second regions or portions of the slide or substrate.

38. The method of claim 37, wherein an observation for the presence or absence of clumping is performed using one or more visualization tools or devices comprising a microscope, a magnifying glass, a loupe or combinations thereof, wherein the visualization tools or devices may be a part of the kit or may be provided separately.

39. The method of claim 38, wherein the visualization tools or devices provide at least a 100× magnification.

40. The method of claim 37, wherein the antigen in the maternal biological sample is a male gender specific hormone, wherein the hormone is testosterone.

41. The method of claim 37, wherein the antibody conjugated to the beads is an anti-testosterone antibody (antiT).

42. The method of claim 37, wherein the presence of clumping in the first region or portion and an absence of clumping in the second region or portion of the slide or substrate is indicative of a male fetus.

43. The method of claim 37, wherein the absence of clumping in the first and second regions or portions of the slide or substrate is indicative of a female fetus.

44. The method of claim 37, wherein the presence of clumping in the second region or portion of the slide or substrate is indicative of a false positive, a testing error or a sample or kit contamination.

45. The method of claim 37, wherein the mammal is a single-fetus human or animal.

46. The method of claim 37, wherein a reaction of the testing reagent and the sample and the reaction of the testing control and the sample may be performed on separate slides or substrates.

47. The method of claim 37, wherein the method may be used to confirm gender determination results done by invasive or non-invasive techniques selected from the group consisting of ultrasound, diagnostic kits, real-time qPCR of maternal plasma, sonography, amniocentesis, chorionic villus sampling (CVS), and any combinations thereof.

48. A testing kit for non-invasive determination of fetal gender in a mammal comprising:
a tube, vial or any other suitable container for receiving one or more maternal biological samples;
a tube or a vial comprising a first testing reagent, wherein the first testing reagent comprises one or more beads conjugated with an antibody, wherein the antibody is capable of binding with an antigen in the maternal biological sample;
a tube or a vial comprising a second testing reagent, wherein the second testing reagent comprises the antibody conjugated with a colored dye, wherein the colored dye is capable of being bleached following reaction of the antibody with the antigen in the maternal biological sample thereby determining the fetal gender;
one or more vials comprising testing controls, wherein the testing controls comprise one or more conjugated beads in a buffer, saline or water, one or more unconjugated beads with the antigen, and or more conjugated beads in a buffer, saline or water with the antibody conjugated with the dye;
one or more slides or other solid substrates for reacting the maternal biological sample with the testing reagents and the testing controls; and
a manual, a booklet, a leaflet or any combinations thereof detailing usage instructions of the testing kit.

49. The testing kit of claim 48, wherein the testing kit is used along with one or more visualization tools or devices comprising a microscope, a magnifying glass, a loupe or combinations thereof, wherein the visualization tools or devices may be a part of the kit or may be provided separately.

50. The testing kit of claim 49, wherein the visualization tools or devices provide at least a 100x magnification.

51. The testing kit of claim 48, wherein the maternal biological sample is selected from the group consisting of serum, urine, blood, and plasma.

52. The testing kit of claim 48, wherein the antigen in the maternal biological sample is a male gender specific hormone, wherein the hormone is testosterone.

53. The testing kit of claim 48, wherein the antibody conjugated to the beads is an anti-testosterone antibody (antiT).

54. The testing kit of claim 48, wherein a presence of testosterone in the maternal biological sample leads to a bleaching of a color of the dye coated antibody from red to white on the slide or the solid substrate, wherein the bleaching is observed using the one or more visualization tools or devices.

55. The testing kit of claim 48, wherein a retention of the red color or a change to a color other than white is indicative of a female fetus.

56. The testing kit of claim 48, wherein the mammal is a single-fetus human or animal.

57. A method for determining fetal gender in a mammal comprising the steps of:
providing a testing kit comprising:
a tube, vial or any other suitable container for receiving one or more maternal biological samples, wherein the biological sample comprises blood, serum, urine, plasma or combinations thereof;
a tube or a vial comprising a first testing reagent, wherein the first testing reagent comprises one or more beads conjugated with an antibody, wherein the antibody is capable of binding with an antigen in the maternal biological sample;
a tube or a vial comprising a second testing reagent, wherein the second testing reagent comprises the antibody conjugated with a colored dye, wherein the colored dye is capable of being bleached following reaction of the antibody with the antigen in the maternal biological sample thereby determining the fetal gender;
one or more vials comprising testing controls, wherein the testing controls comprise one or more conjugated beads in a buffer, saline or water, one or more unconjugated beads with the antigen, and or more conjugated beads in a buffer, saline or water with the antibody conjugated with the dye;
one or more slides or other solid substrates for reacting the maternal biological sample with the testing reagents and the testing controls; and
a manual, a booklet, a leaflet or any combinations thereof detailing usage instructions of the testing kit;
obtaining the one or more maternal biological samples;
adding the first testing reagent to a first region or portion of the slide or the solid substrate;
adding the one or more biological samples to the first region or portion of the slide or substrate comprising the first testing reagent to form a mixture;
incubating the mixture for a specified period of time;
adding the second testing reagent to the first region or portion of the slide comprising the mixture;
adding the one or more testing controls to a second region or portion of the slide or the solid substrate;
adding the one or more biological samples to the second region or portion of the slide or substrate comprising the testing controls to form a mixture;
adding the second testing reagent to the second region or portion of the slide comprising the mixture; and
observing for a bleaching of color of the second testing reagent in the first and second regions or portions of the slide or substrate.

58. The method of claim 57, wherein an observation for of the bleaching of color of the second testing reagent is performed using one or more visualization tools or devices comprising a microscope, a magnifying glass, a loupe or combinations thereof, wherein the visualization tools or devices may be a part of the kit or may be provided separately.

59. The method of claim 58, wherein the visualization tools or devices provide at least a 100× magnification.

60. The method of claim 57, wherein the antigen in the maternal biological sample is a male gender specific hormone, wherein the hormone is testosterone.

61. The method of claim 57, wherein the antibody conjugated to the beads is an anti-testosterone antibody (antiT).

62. The method of claim 57, wherein the bleaching of color from red to white of the second testing reagent in the first region or portion and a retention of a red color of the second testing reagent in the second region or portion of the slide or substrate is indicative of a male fetus.

63. The method of claim 57, wherein the retention of the red color in the first and second regions or portions of the slide or substrate is indicative of a female fetus.

64. The method of claim 57, wherein the bleaching of color from red to white of the second testing reagent in the second region or portion of the slide or substrate is indicative of a false positive, a testing error or a sample or kit contamination.

65. The method of claim 57, wherein the mammal is a single-fetus human or animal.

66. The method of claim 57, wherein a reaction of the testing reagent and the sample and the reaction of the testing control and the sample may be performed on separate slides or substrates.

67. The method of claim 57, wherein the colored dye is phycoerythrin.

68. The method of claim 57, wherein the method may be used to confirm gender determination results done by invasive or non-invasive techniques selected from the group consisting of ultrasound, diagnostic kits, real-time qPCR of maternal plasma, sonography, amniocentesis, chorionic villus sampling (CVS), and any combinations thereof
